SEQUENCE LISTING (9 sequences)

SEQ ID NO: 1 - DNA, 34 bp, Artificial Sequence
  Description of Artificial Sequence Synthetic oligonucleotide
ataacttcgtataatgtatgctatacgaagttat

SEQ ID NO: 2 - DNA, 65 bp, Artificial Sequence
  Description of Artificial Sequence Synthetic primer
agatctaagcttataacttcgtatagcatacattatacgaagttatacaagtttgtacaaaaaag

SEQ ID NO: 3 - DNA, 63 bp, Artificial Sequence
  Description of Artificial Sequence Synthetic primer
ctcgagatcgataacttcgtataatgtatgctatacgaagttataccactttgtacaagaaag

SEQ ID NO: 4 - DNA, 12 bp, Artificial Sequence
  Description of Artificial Sequence Synthetic oligonucleotide
tacgtgatggca

SEQ ID NO: 5 - protein, 4 aa, Artificial Sequence
  Description of Artificial Sequence Synthetic peptide
YVMA

SEQ ID NO: 6 - DNA, 22 bp, Artificial Sequence
  Description of Artificial Sequence Synthetic primer
aatgggcgtggatagcggtttg

SEQ ID NO: 7 - DNA, 24 bp, Artificial Sequence
  Description of Artificial Sequence Synthetic primer
cctacaggtggggtctttcattcc

SEQ ID NO: 8 - DNA, 20 bp, Artificial Sequence
  Description of Artificial Sequence Synthetic primer
cggccccgtgatgaagaaga

SEQ ID NO: 9 - DNA, 23 bp, Artificial Sequence
  Description of Artificial Sequence Synthetic primer
aggcggtgctggatgaagtggta

(19) United States
(12) Patent Application Publication
(10) Pub. No.: US 2009/0165150 A1
(43) Pub. Date: Jun. 25, 2009

(54) DIRECTED COMPLEMENTATION WITH REMOVABLE GENE OF INTEREST

(76) Inventors: Yinghui Zhou, Belmont, MA (US); William Rideout, III, Cambridge, MA (US); Joerg Heyer, Cambridge, MA (US); Murray Robinson, Boston, MA (US)

Correspondence Address: GOODWIN PROCTER LLP;ATTN: PATENT ADMINISTRATOR, EXCHANGE PLACE, 53 STATE STREET, BOSTON, MA, 02109-2881, US

(21) Appl. No.: 11/963,358

(22) Filed: Dec. 21, 2007

(51) Int. Cl. A01K 67/027; C12N 15/87

(52) U.S. Cl. 800/3; 435/462; 800/18

(57) ABSTRACT

The invention provides an improved directed complementation method for generating a conditionally tumorigenic mouse cell. In a directed complementation method, the tumorigenicity of a conditionally tumorigenic mouse cell depends on either the expression of an inducible recombinant oncogene or the expression of a recombinant gene of interest that functionally complements an uninduced recombinant oncogene. The invention provides a method of producing a tumorigenic mouse cell containing an uninduced oncogene, a recombinant gene of interest that functionally complements the uninduced oncogene, and a Cre-ER system capable of excising the recombinant gene of interest. When the Cre-ER system is activated, the recombinant gene of interest is excised. From the effect on the mouse cell it is possible to determine whether the recombinant gene of interest is a tumor maintenance gene.

### FIELD OF THE INVENTION

The field of the invention is molecular biology, oncology, and drug development.

### BACKGROUND OF THE INVENTION

Mouse models of cancer in which primary tumors are driven by specifically engineered oncogenes have become increasingly useful tools in cancer research in recent years. Primary tumors from mouse models are particularly useful in drug development studies, as well as in basic research. Such primary tumors undergo neoplastic transformation through the spontaneous acquisition of different mutations that result in similar or indistinguishable tumor phenotypes, e.g., breast carcinoma. This genotypic variation from tumor to tumor mimicks the genetic variation observed among naturally occurring tumors. Due to such genotypic variation, a given drug may be efficacious against one tumor but not against another tumor of the same phenotype. This differential drug response is potentially useful for predicting human drug response based on animal models.

Recently, an efficient method for generating a tumorigenic mouse cell, i.e., primary tumor material whose tumorigenicity depends on a recombinant gene of interest, has been published (Robinson et al., U.S. Patent Publication No. US 2006/0228302). This method, known as “directed complementation,” facilitates production of tumor material in which tumorigenicity depends on a pre-selected gene of interest, i.e., a target gene.

### SUMMARY OF THE INVENTION

The invention provides an improvement on the directed complementation method for generating a tumorigenic mouse cell as disclosed in U.S. Patent Publication No. 2006/0228302. The invention provides a method of producing a tumorigenic mouse cell, the tumorigenicity of which depends on expression of a recombinant gene of interest. The method includes the steps of:
(a) providing a conditionally tumorigenic mouse cell comprising (i) one or more mutations such that both alleles of an endogenous tumor suppressor gene are absent or nonfunctional, (ii) a gene construct encoding a Cre-ER fusion protein, wherein the gene construct is operatively linked to an endogenous Rosa26 promoter, and (iii) a recombinant oncogene operably linked to an inducible promoter, wherein (1) expression of the recombinant oncogene results in tumorigenicity of the conditionally tumorigenic mouse cell, and (2) the inducible promoter is in the uninduced state; and
(b) introducing into the cell a gene of interest flanked by loxP sites, wherein the gene of interest functionally complements the recombinant oncogene thereby restoring tumorigenicity of the cell without expression of the recombinant oncogene.

The present invention improves the directed complementation technology by adding to the system: (1) a Cre-ER gene operably linked to an endogenous promoter in the conditionally tumorigenic cell (step (a)); and (2) a pair of loxP sites flanking the recombinant gene of interest (step (b)). The conditionally tumorigenic cell can be obtained from a genetically engineered mouse as described, for example, in U.S. Patent Publication No. 2006/0228302, except that the Cre-ER gene is targeted to an endogenous Rosa26 locus in the ES cells used to make the chimeric mouse. The subsequently introduced gene of interest is flanked by loxP sites that permit excision of the gene of interest by the Cre-ER gene product.

Surprisingly, we discovered that a Cre-ER transgene randomly inserted under the control of a strong promoter yielded unsatisfactory results. Overexpression of the Cre-ER fusion protein, in vitro, resulted in unwanted Cre-dependent excision of the gene of interest (flanked by a pair of loxP sites), in the absence of an exogenous antiestrogen. In these experiments, a Cre-ER fusion protein was transfected into conditionally tumorigenic cells. Cre-dependent excision was tested using PCR analysis with two primers flanking the loxP sites. The two PCR primers flanking the loxP sites are sufficiently far apart that no amplification is detected when the recombinant gene of interest is present between the loxP sites (i.e., the unexcised from), but a single PCR product is readily detected following excision of the recombinant gene of interest. In these experiments, a single PCR product was observed in the absence of exogenous antiestrogen indicating unwanted recombination between the loxP sites. The results suggest that, in this system, the Cre activity was leaky.

To solve the problem of insufficiently tight regulation of Cre activity (“leaky” Cre activity), the present invention involves targeted insertion of the Cre-ER construct so its expression is driven by an endogenous Rosa26 promoter that gives ubiquitous expression of the Cre-ER fusion protein at a low-to-moderate level (Zambrowicz et al., 1997, Proc. Nat'l. Acad. Sci. 94:3789-94; Seibler et al., 2003, Nucleic Acids Res., 31:e12). Such targeted insertion, and the resulting ubiquitous expression of the Cre-ER gene at a low-to-moderate level, achieves tight, reliable regulation of Cre activity. This allows controlled excision of the gene of interest only in the presence of an exogenous antiestrogen, which binds to the ER moiety of Cre-ER fusion protein, thereby activating the Cre moiety of the Cre-ER fusion protein.

Examples of tumor suppressor genes that can be usefully knocked out in the foregoing mouse cells, include, for example, Rb, P53, INK4a, PTEN, LATS, Apaf1, Caspase 8, APC, DPC4, KLF6, GSTP1, ELAC2/HPC2, NKX3.1, ATM, CHK2, ATR, BRCA1, BRCA2, MSH2, MSH6, PMS2, Ku70, Ku80, DNA/PK, XRCC4, Neurofibromatosis Type 1, Neurofibromatosis Type 2, Adenomatous Polyposis Coli, the Wilms tumor-suppressor protein, Patched and FHIT. Tumor suppressor genes preferably knocked out in the mouse cells include INK4a, P53, PTEN and Rb, where INK4a is most preferred.

Examples of recombinant oncogenes useful in the present invention include Her2, KRAS, HRAS, NRAS, EGFR, FGFR1, FGFR2, FGFR3, FGFR4, MDM2, TGF-β, RhoC, AKT, c-myc, β-catenin, PDGF, C-MET, P13K-100α, CDK4, cyclin B1, cyclin D1, estrogen receptor gene, progesterone receptor gene, ErbB1, ErbB3, PLK3, KIRREL, ErbB4, TGFα, ras-GAP, Shc, Nck, Src, Yes, Fyn, Wnt, Bcl2, PyV MT antigen, and SV40 T antigen. Preferred oncogenes include Her2, C-MET, P13K-CA and AKT, where Her2 and KRas are most preferred.

As used herein, the term “conditionally tumorigenic mouse cell” means a mouse cell in which tumorigenicity depends on induction of expression of a recombinant oncogene.

As used herein, the term “Cre-ER gene” means a gene encoding a fusion protein comprising a Cre recombinase moiety and the ligand-binding domain of an estrogen receptor (“ER”) that is activated by an exogenous estrogen, but is not activated by any endogenous mouse estrogen (i.e., 17B-estradiol). In certain embodiments, the Cre-ER gene encodes a hinge region between the Cre domain and the ER domain (see, e.g., Chambon et al., supra). Preferred estrogen receptors are mutated or modified estrogen receptors such as ER^{T} or ER^{T2}. The ER^{T} domain comprises the ligand binding domain (amino acids 282-595) of the human estrogen receptor carrying a G521R mutation and the ER^{T2} domain comprises the ligand binding domain (amino acids 282-595) of the human estrogen receptor carrying three mutations (G400V/M543A/L544A) as described by Chambon et al., supra, and Siebler et al., supra. The single mutation in the ER^{T} domain reduces affinity of natural ligand, i.e., 17B-estradiol, by approximately 1000 fold, without adversely affecting the binding of exogenous antiestrogens (e.g., tamoxifen and 4-hydroxytamoxifen) (see, e.g., Chambon et al., supra). The triple mutation in the ER^{T2} further enhances the sensitivity of the mutated estrogen receptor by 10 fold to exogenous antiestrogens compared to the ER^{T} domain (see, e.g., Siebler et al., supra).

As used herein, the term “loxP site” means a 34 base pair nucleic acid sequence comprising two 13 base pair palindromes separated by an asymmetric 8 base pair core sequence, e.g., ATAACTTCGTATAATGTATGCTATACGAAGTTAT (SEQ ID NO: 1), and includes nucleic acid sequence derivatives that are active in Cre-mediated recombination as described by Hoess et al., 1986, Nucleic Acids Res. 14:2287-2300 and Sheren et al., 2007, Nucleic Acids Res., 35:5464-5473 (doi: 10.1093/nar/gkm604).

As used herein, the term “tetracycline-dependent promoter system” means a gene expression system that includes a tetracycline-dependent promoter and either a reverse tetracycline-controlled transactivator (rtTA) or a tetracycline-controlled transactivator (tTA) described by Gossen et al. (1995, Science, 268:1766-1769) and Gossen et al. (1992, Proc. Natl. Acad. Sci., 89:5547-5551), respectively. Complete tetracycline-regulated mammalian expression systems are available commercially, e.g., T-Rex™ (Invitrogen, Carlsbad, Calif.) and Tet-Off® and Tet-On® Gene Expression Systems (Clontech, Mountain View, Calif.). In tTA expression systems, gene expression is turned off when tetracycline (tet) or a functional tetracycline analog such as doxycycline (dox) is added to the system, and turned on when tet or a tet analog is removed. In rtTA expression systems, expression is turned on when tet or a tet analog is added to the system and turned off when the tet or tet analog is removed.

Unless otherwise defined, all technical and scientific terms used herein have the same meaning as commonly understood by one of ordinary skill in the art to which the invention pertains. In case of conflict, the present specification, including definitions, will control. All publications, patents and other references mentioned herein are incorporated by reference in their entirety for all purposes.

Although methods and materials similar or equivalent to those described herein can be used in the practice or testing of the invention, the preferred methods and materials are described below. The materials, methods and examples are illustrative only, and are not intended to be limiting. Other features and advantages of the invention will be apparent from the detailed description and from the claims.

### BRIEF DESCRIPTION OF THE DRAWINGS

FIG. 1 (prior art) is a schematic drawing illustrating the basic concept of directed complementation as described in U.S. Patent Publication No. 2006/0228302.

FIG. 2 is a schematic drawing illustrating the basic principle of how the tumorigenic mouse cells produced by the method of the present invention are used (i) to confirm that restoration of tumorigenicity in the cell is in fact caused by the putative complementing gene, and (ii) to determine whether the gene of interest, i.e., the complementing gene, is a tumor maintenance gene.

### DETAILED DESCRIPTION OF THE INVENTION

The present invention provides a system for “loss-of-function” confirmation that restoration of tumorigenicity of a cell by the directed complementation method is, in fact, caused by the gene of interest, i.e., the putative complementing gene. Loss of function is triggered by activating the Cre-ER fusion protein, which results in loss (i.e., excision) of the putative complementing gene. This results in loss of tumorigenicity, if the putative complementing gene is in fact restoring tumorigenicity to the cell by functionally complementing the inducible oncogene that is switched off during directed complementation.

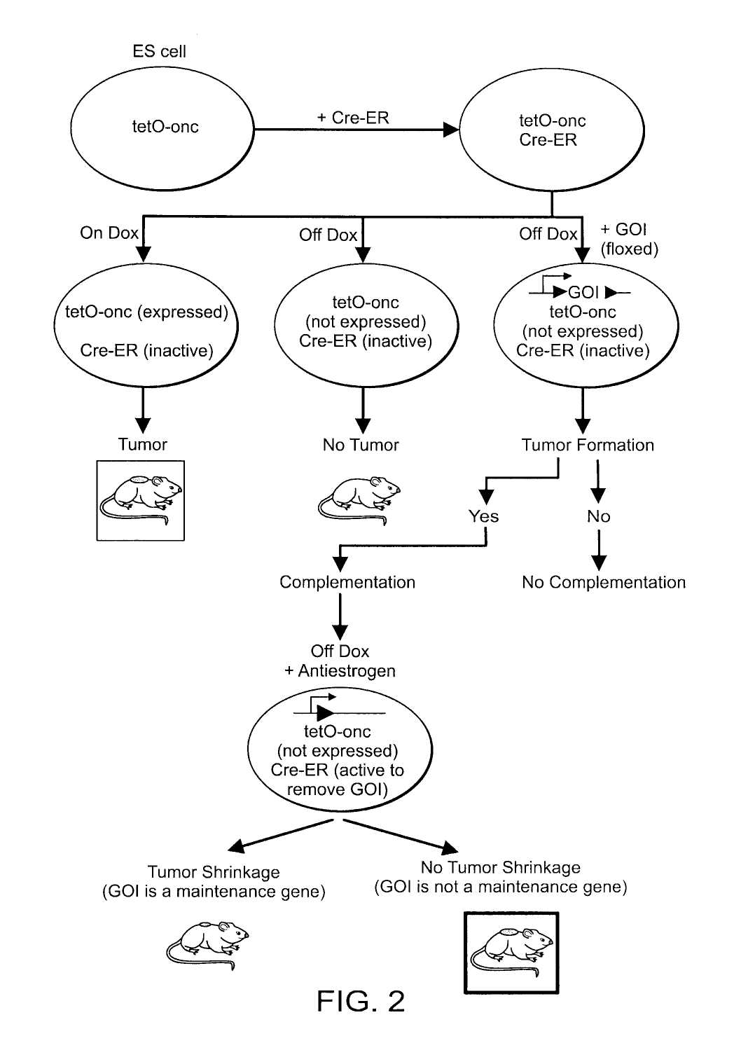

The present invention also provides a mouse cell that can be used to determine whether the complementing gene is a tumor maintenance gene, as opposed to a gene that is necessary for tumor induction but unnecessary for continued viability and growth of the tumor. In general, such determination is made by comparing the effect of administering the exogenous antiestrogen, e.g., tamoxifen or 4-hydroxytamoxifen, to a host mouse in a xenograft experiment (FIG. 2; Example 5) at the time of xenograft injection with the effect of administering the antiestrogen after a tumor has formed. If the antiestrogen prevents tumor formation, but does not cause shrinkage of an existing tumor, the complementing gene is not necessary for continued viability and growth of the tumor, and thus is not a tumor maintenance gene. If the antiestrogen prevents tumor formation and also causes shrinkage of an existing tumor, the complementing gene is necessary for continued viability and growth of the tumor, and thus is a tumor maintenance gene.

Directed complementation provides a practical method for efficiently obtaining a tumorigenic mouse cell, the tumorigenicity of which depends on a recombinant gene of interest. In general, neoplastic transformation results from an accumulation of mutations, rather than a single mutation. Therefore, merely transfecting a wild type mouse cell, e.g., a mammary epithelial cell, with a recombinant oncogene of interest would not be sufficient to yield a tumorigenic cell. Instead, mice are engineered to express a recombinant oncogene in a tissue-specific or organ-specific manner, in the target tissue or organ, and to lack expression of a tumor repressor gene. See, e.g., U.S. Pat. No. 6,639,121; and WO 2005/020683. Following a latency period during which mutations accumulate, tumors arise spontaneously in the target tissue or organ.

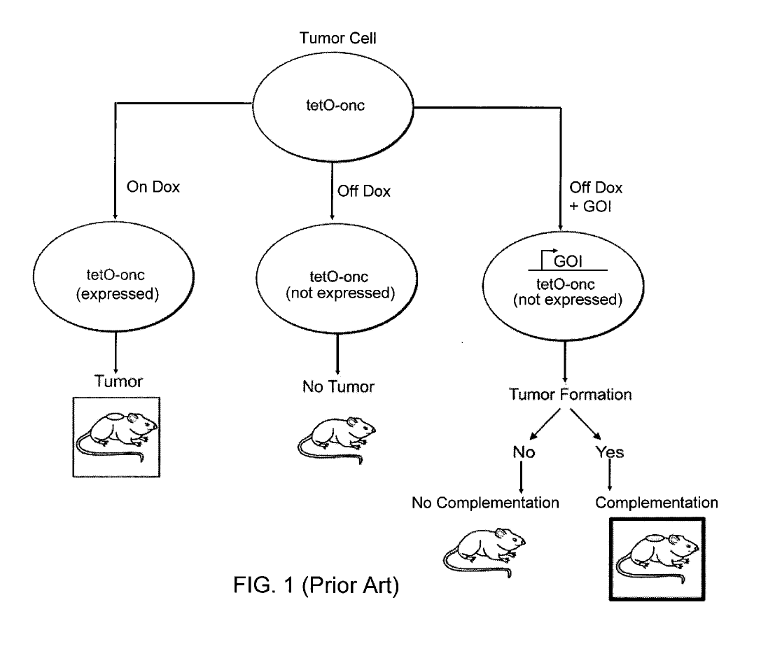

These spontaneous tumors generally are dependent upon expression of the inducible recombinant oncogene. When the inducer is not provided to the animal, the tumor regresses, and the cells of the target tissue or organ become non-tumorigenic. However, the cells of the target tissue or organ are only one mutation away from being tumorigenic. All that is necessary to restore tumorigenicity is: (1) expression of the inducible recombinant oncogene, or (2) expression of a gene of interest that functionally complements, i.e., substitutes for, the recombinant oncogene (see FIG. 1).

As illustrated in FIG. 1, the tumor cell comprises a recombinant oncogene (onc) operatively linked to a tetracycline inducible promoter (tetO). Tumorigenicity of the tumor cell may be restored following administration of an inducer, such as doxycycline (“on dox” in FIG.1), which results in the expression of the recombinant oncogene. This result is compared to no expression of the recombinant oncogene in the absence of the inducer (“off dox” in FIG. 1). Alternatively, tumorigenicity of the tumor cell can be restored through expression of a recombinant gene of interest (“GOI”) that functionally complements the recombinant oncogene (FIG. 1).

The cells at this point are said to be “conditionally tumorigenic cells.” A nucleic acid encoding the recombinant gene of interest that functionally complements the recombinant oncogene is introduced into the conditionally tumorigenic cell by any suitable method. Thus, genetic complementation is achieved in a directed manner, so as to obtain a tumorigenic cell in which tumorigenicity is driven by a pre-selected gene of interest. Typically, the gene of interest is a potential therapeutic target for anti-cancer molecules in a drug development program.

A single source of conditionally tumorigenic cells, e.g., a single primary tumor, can be used to generate numerous lines of primary tumor material, with the tumorigenicity of each line being dependent on a different, pre-selected, cancer-related gene of interest. A second source of conditionally tumorigenic mouse cells, dependent on a second oncogene, can be used to generate additional lines of primary tumor material, with the tumorigenicity of each line being dependent on a new set of pre-selected, cancer-related genes. A third source of conditionally tumorigenic mouse cells, dependent on a third oncogene, can be used, and so forth.

When a single source of conditionally tumorigenic cells is used to generate different lines of primary tumor material by introducing different genes of interest, the effects of the different genes of interest on the tumors, with and without drug treatment, can be evaluated in exactly the same genetic background. In contrast, if separate mouse models were independently engineered to incorporate the same genes of interest and spontaneous tumors were generated, the genetic background of the gene of interest would be different in each model. Consequently, the type of comparison possible with the present invention would not be possible using the separately engineered models.

Looking at the genetic background question in the opposite way, a given gene of interest can be introduced separately into conditionally tumorigenic cells from multiple tumors. This will allow the effect of a given gene of interest, with and without drug treatment, to be evaluated in different genetic backgrounds.

For any given conditionally tumorigenic cell, the inducible recombinant oncogene is known. With knowledge of the inducible recombinant oncogene in hand, the skilled person can identify one or more genes of interest that will functionally complement the inducible recombinant oncogene. For example, the receptor tyrosine kinase, Her2/Neu/ErbB2 is known to be important for the viability of a subset of human breast cancers. Much is known about the downstream mediators of Her2, and this information has been summarized by publicly available sources, e.g., Biocarta. From such information, the skilled person can predict a useful number of complementing genes with a reasonable expectation of success. For example, genes expected to complement Her2 would include ErbB1/EGFR, ErbB3, PLK3, KIRREL, PI3K, Ras, Akt, Raf, Erk1 and Erk2.

One of the genes expected to complement Her2 is the ErbB3 gene, also known as HER3, MDA-BF-1 and MGC88033, which encodes a member of the epidermal growth factor receptor (EGFR) family of receptor tyrosine kinases. The ErbB3 membrane-bound protein has a neuregulin binding domain but not an active kinase domain. It therefore can bind the neuregulin ligand but not convey the signal into the cell through protein phosphorylation. However, it does form heterodimers with other EGF receptor family members which do have kinase activity. Heterodimerization leads to the activation of pathways which lead to cell proliferation or differentiation. Amplification of this gene and/or overexpression of its protein have been reported in numerous cancers, including prostate, bladder, and breast tumors (see, e.g., van der Horst et al., 2005, Int J Cancer 115:519-527; Holbro et al., 2003, Proc Natl Acad Sci USA. 100:8933-8938).

Another gene expected to complement Her2 is the gene designated PLK3 (polo-like kinase 3), also known as CNK, FNK, PRK, and 1_{—}44702099GP201. The PLK3 gene encodes a putative serine/threonine kinase and is a member of the “polo” family of serine/threonine kinases, which is likely to play a role in cell cycle progression and tumorigenesis (see, e.g., Li et al., 1996, J Biol Chem 271:19402-19408). High expression of PLK3 has been detected in cancers of the bladder, breast, colon, ovary, pancreas and lung (see, e.g., Dai et al., 2000, Genes Chromosomes Cancer 27:332-336; Li et al., 1996, J Biol Chem 271:19402-19408).

Another gene expected to complement Her2 is the gene designated KIRREL, also known as NEPH1, FLJ10845, LOC348416 or 1_{—}154843723, which encodes a protein member of the nephrin-like protein family and contains an immunoglobulin domain. KIRREL expression is elevated in chondrosarcoma, glioblastomas, including glioblatomas expressing mutant activated EGFR, astrocytomas and medulloblastomas, pancreatic adenocarcinoma, breast carcinomas and colon adenocarcinoma, and melanomas.

Another gene known to be involved in tumorigenesis is mTOR kinase. Genes expected to complement mTOR kinase would include PI3Kinase, Akt1, Rheb, and S6Kinase. Another gene known to be involved in tumorigenesis is KRas. Genes expected to complement KRas would include Raf, Mekk1, Mekk2, Erk1, Erk2 and Jnk1.

In some cases, the skilled person will identify the recombinant gene of interest first, and then work “backwards” to identify an oncogene that would be functionally complemented by the gene of interest, based on knowledge available to one of skill in the art regarding signal transduction pathways and biochemical pathways that operate in particular types of cancer. That oncogene would be chosen as the inducible recombinant oncogene to use in producing the conditionally tumorigenic cells.

The gene of interest introduced into the conditionally tumorigenic mouse cells can be a mouse gene. In some embodiments of the invention, however, it may be preferable to introduce the human ortholog of the gene of interest into the conditionally tumorigenic mouse cells. An advantage of employing the human gene is that when the resulting tumors are subsequently used in drug development studies, the test compounds will be tested against the human target molecules rather than the mouse orthologs. Working with the human molecules will eliminate one potential source of unpredictability relatively early in the drug development process.

When embryonic stem cells containing the desired genetic modifications, i.e., an inducible recombinant oncogene and a second gene that confers a predisposition to develop cancer, are injected into an early stage mouse embryo, e.g., a blastocyst, the result is a chimeric mouse. See, e.g., WO 2005/020683.

While preserving (in some percentage of its cells) the same genetic design as a conventional germline transgenic mouse, a chimeric mouse provides certain advantages. For example, to generate a conventional germline transgenic melanoma model as described in Chin et al., 1999, Nature 400:468-472, one would have to breed three animal lines with four respective genetic alterations, i.e., homozygous INK4a null mutation, a Tyr-rtTA transgene, and a tetO-H-ras transgene, to obtain a transgenic animal with all four genetic alterations. This extensive breeding requires a considerable amount of time. In contrast, a chimeric melanoma model requires no breeding. One needs only to establish ES cells with all four genetic alterations and inject them into a blastocyst, which develops into an intact animal upon transplantation into the uterus of a surrogate mother. The average time saved can be as much as one year. A second advantage is that in a chimeric mouse, spontaneous tumors develop in an environment that includes normal cells. This resembles the natural disease situation more closely than the cellular environment in a germline transgenic mouse, where every cell is genetically modified.

A useful ES cell line can be established by introducing more than two nucleic acid constructs into an ES cell concurrently or sequentially, where each construct may contain one or more genetic elements that will cause genetic alterations of the host genome. These genetic elements can also be inserted into one single vector, e.g., a BAC, PAC, YAC or MAC vector.

Targeted genetic alterations can introduce a desired change to a specific location in an endogenous gene. Examples of the changes include a null (knock out) mutation in a tumor suppressor gene locus or an activating mutation (knock in) to a cellular oncogene. For instance, one can replace a coding or regulatory region of a tumor suppressor gene with a selectable marker gene flanked by a pair of LoxP sites; or insert a dominant negative mutation into a tumor suppressor gene; or replace the native promoter of a cellular oncogene with a constitutive or inducible promoter; or inserting an activating mutation into a cellular oncogene (see, e.g., Johnson et al., 2001, Nature 410:1111-1116). Such a genetic alteration can be accomplished by homologous recombination. In a nucleic acid construct used for homologous recombination, the genetic alteration to be introduced into the host genome is flanked by sequences homologous to the targeted genomic region.

Oncogenes useful for engineering mice (germline transgenic or chimeric) to develop inducible spontaneous tumors include KRAS, HRAS, NRAS, epidermal growth factor receptor (EGFR), fibroblast growth factor receptor (e.g., FGFR1, FGFR2, FGFR3, FGFR4), MDM2, TGF-β, RhoC, AKT family members, myc (e.g., c-myc), β-catenin, PDGF, C-MET, PI3K-CA, CDK4, cyclin B1, cyclin D1, estrogen receptor gene, progesterone receptor gene, Her2 (also known as neu or ErbB2), ErbB1, ErbB3, ErbB4, TGFα, ras-GAP, Shc, Nck, Src, Yes, Fyn, Wnt, Bcl2 anti-apoptotic family members, and viral proteins such as PyV MT and SV40 T antigens. Activating mutations of these oncogenes, e.g., Her2V664E, K-RasG12D, and β-cateninΔ131, also can be used.

Tumor suppressor genes whose inactivation is useful for engineering mice (germline transgenic or chimeric) to develop inducible spontaneous tumors include Rb, P53, INK4a, PTEN, LATS, Apaf1, Caspase 8, APC, DPC4, KLF6, GSTP1, ELAC2/HPC2 or NKX3.1. Other examples of tumor suppressor genes are those involved in DNA damage repair (e.g., ATM, CHK2, ATR, BRCA1, BRCA2, MSH2, MSH6, PMS2, Ku70, Ku80, DNA/PK, XRCC4 or MLH1), and cell signaling and differentiation (e.g., Neurofibromatosis Type 1, Neurofibromatosis Type 2, Adenomatous Polyposis Coli, the Wilms tumor-suppressor protein, Patched or FHIT). In addition to targeted mutation, tumor suppressor genes can be inactivated by an antisense RNA, RNA interference (RNAi), or ribozyme agent expressed from a construct stably integrated into the host genome.

Mice engineered to develop inducible spontaneous tumors for use as a source of conditionally tumorigenic cells can be developed from ES cells that contain an introduced active oncogene as well as one or more inactivated endogenous tumor suppressor gene(s). For example, the ES cells can contain genetic alterations that result in the expression of an activated form of EGFR (designated as EGFR*) in combination with reduced p16^{INK4a} or p19^{ARF} expression (e.g., genetic alterations that produce an EGFR*⁺ and INK4a/ARF^{-/-} genotype); genetic alterations that result in PDGF expression in combination with reduced p53 expression (e.g., genetic alterations that produce a PDGF⁺and p53^{-/-} genotype); genetic alterations that result in TGF-α expression in combination with reduced p53 expression (e.g., genetic alterations that produce a TGFα⁺ and p53^{-/-} genotype); and genetic alterations that result in reduced PTEN expression and reduced p16^{INK4a} or p19^{ARF} expression (e.g., genetic alterations that produce a PTEN^{-/-} and INK4a/ARF^{-/-} genotype).

Various vectors are useful for doing genetic manipulations and obtaining the genetically modified mouse cells necessary for practicing the present invention. Suitable vectors can be derived from plasmids, retroviruses, adenoviruses, or lentiviruses. Expression vectors typically include various genetic elements operatively linked to a polypeptide-encoding heterologous nucleic acid insert. Examples of such genetic elements are those that affect transcription and RNA processing, e.g., operators, silencers, promoters and enhancer elements, transcription termination signals, RNA splicing signals and polyadenylation signals. Other signals affect translation, e.g., ribosomal consensus sequences. The use of such expression control elements, including those that confer constitutive or inducible expression, and developmental or tissue-specific expression are known in the art.

The vectors can be introduced into mouse cells, including ES cells and tumor cells, by various methods, including cell fusion (e.g., spheroplast fusion), liposome fusion (transposomes), conventional nucleic acid transfection methods such as calcium phosphate precipitation, electroporation, microinjection, or infection by viral vectors. Various methods can be used to screen for cells that have stably incorporated the desired genetic alterations. Such methods include detection of drug resistance where a drug selection marker gene (e.g., a neomycin-resistant gene, a puromycin-resistant gene, or a hygromycin-resistant gene) is co-introduced; detection of fluorescence or bioluminescence emission where a fluorescence or bioluminescence marker gene (e.g., a gene encoding a green, yellow, blue or red fluorescent protein, and Luciferase genes) is co-introduced; polymerase chain reaction (PCR); and Southern blot analysis.

Recombinant genes, e.g., a recombinant oncogene or a gene of interest, can be placed under the control of an inducible promoter such as the tetracycline-regulated promoter system as described in e.g., WO 01/09308. Complete tetracycline-regulated mammalian expression systems are available commercially, e.g., T-Rex™, Invitrogen, Carlsbad, Calif. When using such a system, the inducing agent (e.g., tetracycline or doxycycline) can be administered conveniently in food or drinking water. Other useful inducible promoters include the metallothionine promoter, the IPTG/lacI promoter system, the ecdysone promoter system, and the Gal4/UAS system, which is available commercially, e.g., GeneSwitch™, Valentis, Inc., Burlingame, Calif. The “lox stop lox” system can be used to delete inhibitory sequences, thereby irreversibly inducing expression of a particular gene to commence in a particular tissue at a particular point in development of the mouse. For a discussion of inducible promoters in transgenic mouse cells and transgenic mice, see Lewandoski, 2001, Nature Rev. 2:743-755.

Recombinant genes introduced into mouse cells can be placed under the control of a tissue-specific promoter, such as a tyrosinase promoter or a TRP2 promoter in the case of melanoma cells and melanocytes; an MMTV or WAP promoter in the case of breast cells and/or cancers; a Villin or FABP promoter in the case of intestinal cells and/or cancers; a PDX promoter in the case of pancreatic cells; a RIP promoter in the case of pancreatic beta cells; a Keratin promoter in the case of keratinocytes; a Probasin promoter in the case of prostatic epithelium; a Nestin or GFAP promoter in the case of central nervous system (CNS) cells and/or cancers; a Tyrosine Hydroxylase, S100 promoter or neurofilament promoter in the case of neurons; the pancreas-specific promoter described in Edlund et al., 1985, Science 230:912-916; a Clara cell secretory protein promoter in the case of lung cancer; and an Alpha myosin promoter in the case of cardiac cells.

Any ES cell lines that provide adequate chimerism can be used. Useful cell lines include E14.1, WW6, CCE, J1, and AB1. See also Alex Joyner, Ed., Gene Targeting, A Practical Approach, Chapter 4 (Virginia Papaioannou), Oxford Press, 2^{nd} Ed., (2000). In general, when chimeric mice are used, the extent of chimerism is not critical. Chimerism of 10% to 90% is preferred.

As used herein, “chimeric” means chimeric in terms of ontogeny. Accordingly, a chimeric mouse is a mouse that has grown, i.e., developed, directly from a multicellular embryo into which at least one genetically modified ES cell has been injected or aggregated. A chimeric mouse is to be distinguished from a morphologically developed mouse that has received a xenograft, e.g., an organ graft, a tissue graft, or a tumor graft from another animal.

A chimeric mouse can be generated by introducing ES cells containing into a host embryo. This can be done, for example, by blastocyst injection or aggregation with earlier stage pre-implantation embryos (e.g., eight-cell embryo). The embryo is subsequently transferred into a surrogate mother for gestation. Chimerism in the born animal can be determined by phenotype (such as fur color, if the host embryo and the ES cells are derived from animal strains of different fur colors), PCR, Southern blot analysis, or biochemical or molecular analysis of polymorphic genes (such as glucose phosphate isomerase). To facilitate identification of chimeric mice having a desired genetic alteration, one can co-introduce a detectable reporter gene and the desired genetic alteration into the ES cells. Exemplary reporter genes include those that encode a fluorescent protein such as a green fluorescent protein, a yellow fluorescent protein, a blue fluorescent protein, or a luminescent protein such as luciferase or β-galactosidase.

The chimeric mice provide flexibility in developing different disease models. For example, ES cell lines can be established for different cancer models by knocking out both alleles of a tumor suppressor gene (e.g., Ink4a/ARF, p53 or PTEN) and introducing a reporter gene (e.g., luciferase), a tissue-specific reverse tetracycline transactivator gene (i.e., MMTV-rtTA) and an oncogene of choice (e.g., Akt, Her2V664E, Her2, Bcl2, K-Ras and Cyclin D1) under the control of a promoter regulated by reverse tetracycline transactivator (rtTA).

Introduction of the recombinant gene of interest into the conditionally tumorigenic mouse cell can be by any suitable method. Various methods are known in the art, e.g., retroviral vectors, lentiviral vectors, lipofection and electroporation. A preferred method is transduction using a retroviral vector. Preferably, the tumor cells are removed from a donor mouse, subjected to a transduction procedure (or other method of introducing the recombinant gene of interest), and placed into a recipient mouse within 48 hours. It has been found that putting the cells back into an animal within 48 hours preserves the inducibility of the oncogene. Loss of inducibility sometimes is observed, when the cells are maintained in vitro for longer periods.

For a general discussion and details of the Cre-ER/loxP/exogenous antiestrogen system system, see, e.g., Chambon et al., U.S. Pat. No. 7,112,715. See also, O'Neal et al., 2007, Methods in Molecular Biology 366:309-320; and Seibler et al., supra. In exemplary embodiments, the Cre-ER fusion protein is a Cre-ER^{T2} fusion protein, as described in Chambon et al., supra. In the Cre-ER fusion protein, the Cre recombinase moiety is activated in the presence of exogenously added antiestrogen. Exemplary antiestrogens include, for example, tamoxifen, 4-hydroxy-tamoxifen (OHT), RU486, ICI 164384 and ICI 182780.

Activation of Cre recombinase by an antiestrogen in the present invention results in the excision of the recombinant gene of interest and, thus, can be used to determine whether restoration of tumorigenicity of the cell is caused by the putative complementing gene. If a recombinant gene of interest functionally complements the uninduced oncogene, then loss of function of the complementing gene following excision will result in a loss of tumorigenicity of the cell. Alternatively, if the recombinant gene of interest does not functionally complement the uninduced oncogene, then the loss of function of the complementing gene following excision likely will not effect the tumorigenicity of the cell.

In an exemplary embodiment, as illustrated in FIG. 2, an exemplary ES cell expresses a recombinant oncogene (onc) operatively linked to an inducible promoter (tetO) and a Cre-ER fusion protein that is conditionally active upon the administration of an antiestrogen. To avoid confusion or ambiguity the present inventors use the term “activation” in reference to the Cre-ER fusion protein, instead of the term “induction,” which sometimes is used in the scientific literature (e.g., Seibler et al., supra). The inventors employ this terminology to indicate that the activity of the Cre-ER fusion protein is controlled at the protein level (conformational change in the Cre-ER protein upon binding to an antiestrogen) rather than at the gene expression level. The skilled person will recognize that the term “inducible Cre,” as sometimes used in the art, has the same meaning as “active Cre” used herein.

As described above for directed complementation (illustrated in FIG. 1), tumorigenicity of the cells can be restored by inducing the expression of the recombinant oncogene (compare “on dox” and “off dox” in FIG. 2). Alternatively, tumorigenicity of the cells may be restored by expression of a complementing gene of interest (“GOI” in FIG. 2) flanked by loxP sites. The term of art when referring to a gene flanked by loxP sites is “floxed.” In certain embodiments, the complementing gene is excised following activation of Cre recombinase by an antiestrogen to confirm that restoration of tumorigenicity is caused by the complementing gene.

The present invention further provides a method for determining whether a complementing gene is a tumor maintenance gene that is necessary for the continued viability and growth of the tumor. The method comprises the steps of: (i) producing a multiplicity of tumorigenic mouse cells, as described herein, where the tumorigenicity of the cells depend on the expression of a recombinant gene of interest; (ii) implanting at least one cell into a host mouse; (iii) obtaining in the mouse a tumor derived from the implanted cells; (iv) administering an exogenous antiestrogen to the at least one mouse; and (v) determining any anti-tumor effects following the loss of expression (i.e., the excision) of the recombinant gene of interest.

In some embodiments of the invention, the antiestrogen is added at the time of xenograft injection (i.e., the implantation of the tumor cells). In other embodiments, the antiestrogen is added only after tumor formation. Administration of antiestrogen is well known in the art, see e.g., Bex et al., 2002, J. Urol., 168:264-2644, Bhatia et al., 2004, J. Pharm. Pharm. Sci., 7:252-259, and Bosenberg et al., 2006, Genesis, 44:262-267. Antiestrogen may be administered topically, orally, or by intraperitoneal injection (IP). In an exemplary embodiment, antiestrogen is administered by intraperitoneal injection. For IP and oral administration, tamoxifen may be dissolved in corn oil or sunflower oil at approximately 10 mg/ml. For topical administration, tamoxifen may be dissolved in ethanol/DMSO or emollient cream. In certain embodiments, mice are treated with 0.1-10 mg per 40 g mouse for IP or oral administration or 0.5-100 mg per 40 g mouse for topical administration.

Tumorigenicity of the cells is monitored following loss of expression (i.e., excision) of the complementing gene (illustrated in FIG. 2). If the administration of the antiestrogen prevents tumor formation and induces tumor regression (shown as “tumor shrinkage” in FIG. 2), the recombinant gene of interest is necessary for continued viability and tumor growth and is a tumor maintenance gene. Alternatively, if the administration of the exogenous antiestrogen prevents tumor formation, but does not induce tumor regression (shown as “no tumor shrinkage” in FIG. 2), the recombinant gene of interest is not necessary for continued tumor growth and is not a tumor maintenance gene. Tumor shrinkage, as used herein, means a reduction in tumor volume, for example, at least a 10%, 20%, 30%, 40%, 50,%, 60,%, 70%, 80%, 90%, 95%, or 98% reduction in tumor volume.

### EXAMPLES

The invention is further illustrated by the following examples. The examples are provided for illustrative purposes only, and are not to be construed as limiting the scope or content of the invention in any way.

### Example 1

### Engineering Chimeric Tumor Models in ES Cells

Chimeric Breast Her2 Model (BH): Ink4a homozygous null ES cells were co-transfected (electroporation) with the following four constructs, as separate fragments: MMTV-rtTA, TetO-Her2^{V664Eneu}, TetO-luciferase and PGK-puromycin, as described in US Patent Publication No. 2006/0228302. Puromycin-resistant cells were genotyped by PCR and Southern blot. Inducibility of the oncogenes in ES cells was analyzed by Northern blot. Three BH model ES cell lines, 31G9, 25A5, and 24C2, have been shown previously to give rise to Doxycycline induced adenocarcinomas in the mammary gland. Cell line 31G9 was selected for further modification.

Chimeric Lung KRAS Model (LK): Ink4a homozygous null ES cells were co-transfected (electroporation) with the following four constructs, as separate fragments: CCSP-rtTA, TetO-KRAS^{G12V}, TetO-luciferase and PGK-puromycin, as described in WO2005/020683. Puromycin-resistant cells were genotyped by PCR and Southern blot analysis. Inducibility of the oncogenes in ES cells was analyzed by Northern blot. Three LK model ES cell lines, 17A8, 17B6 and 17C3, have been shown previously to give rise to Doxycycline induced lung adenocarcinomas. Cell line 17B6 was selected for further modification.

Rosa-CreER targeting construct: The targeting construct was assembled using a commercial kit (RED®/ET® Genebridges, Dresden, Germany) for λ-mediated recombination techniques. For a discussion of these techniques, see, e.g., Muyrers et al., 1999, Nucleic Acids Res. 27:1555-1557. The RED/ET commercial kit contained: (a) the ColE1 origin of replication and ampicillin resistance gene from pUC19; (b) a 3.5 kb fragment clone (from RPCI23 female C57B16 mouse genomic library) which contains the promoter and first exon of the Rosa26 locus and serves as the 5′ homology arm for targeting the Rosa26 locus in the mouse genome; (c) the rabbit beta globin splice acceptor site upstream of the CreERT2 cDNA construct (see, e.g., Indra et al., 1999, Nucleic Acid Res. 27:4324-4327); followed by (d) an SV40 polyA signal sequence; (e) the FRT-PGKgb2neo-FRT eukaryotic/prokaryotic selection cassette from Genebridges which is flanked by FRT recombination sites and provides resistance to G418 and Kanamycin; and (f) a 4.8 kb fragment of the Rosa26 locus subcloned from the RPCI23-324018 BAC to serve as the 3′ homology arm for targeting in mouse embryonic stem cells. Also, at the end of each homology arm, a SwaI restriction endonuclease site was added in order to linearize the targeting plasmid and remove the bacterial ColE1 and Ampicillin resistance genes prior to electroporation of the targeting construct into embryonic stem cells.

BHc and LKc model: The selected ES cells (31G9 for BH and 17B6 for LK) were further transfected with the Rosa-CreER targeting construct described above. The Rosa-CreER targeting construct was used to insert the CreER expression cassette into the Rosa26 locus in the mouse genome in order to have the CreER mRNA expressed under the endogenous Rosa26 promoter. This results in ubiquitous expression of the CreER fusion protein at a moderate level, which provides tight regulation of tamoxifen-induced, Cre-mediated recombination at LoxP sites. Transfection was by an electroporation procedure in which 25 to 50 micrograms of SwaI digested DNA was mixed with 5×10⁶ ES cells in 800 microliters of PBS in a 4 mm cuvette and electroporated by a pulse of 600 volts, 25 microfarads. Targeting efficiency was approximately 10-20% of G418 resistant colonies as assessed by Southern blot analysis of ES cell genomic DNA. The BH Model ES cell lines with CreER targeted into the Rosa26 locus were designated as the BHc Model and the LK model ES cell lines with CreER targeted into the Rosa26 locus were designated as the LKc model.

The Rosa-CreER targeted ES cells were injected into C57BL/6 blastocysts, which were transplanted into pseudo-pregnant female mice for gestation leading to birth of chimeric mice. Among all the lines tested, the BHc model lines 31G9_E4, 31G9_G4 and 31G9_G7, and the LKc model lines 17B6_B7.10 and 17B6_C8.7 were used to produce chimeric mouse cancer models.

### Example 2

### Conditionally Tumorigenic Cells for Directed Complementation

Tumor induction in BHc chimeras: The BHc model ES cell lines were injected into C57/BL6 blastocysts to produce multiple chimeras. Chimerism, as judged by coat color, ranges from 50 to 100%. Same as in the BH model, the mouse mammary tumor virus long terminal repeat (MMTV) drives breast-specific expression of the reverse tetracycline transactivator (rtTA), which activates breast-specific expression of the HER2 oncogene in the presence of Doxycycline. Following induction by doxycycline provided to the mice in their drinking water (2500 ppm), the mice developed mammary tumors with a latency of about 2-4 months. As expected, these tumors exhibited the same characteristics as the tumors from the predecessor BH model as described in WO2005/020683 and U.S. Patent Publication No. 2006/0228302. These tumors are referred to as primary BHc tumors.

BHc Primary Culture: Primary BHc tumors were minced with scalpels and passed through 100 micron Cell Strainers (BD Biosciences) to remove debris. Approximately 100,000 tumor cells were plated in each well of a 6 well cluster plate in 2.5 ml of DMEM medium (containing 10% FBS, 50 U/ml penicillin, 50 μg/ml streptomycin and 2 μg/ml doxycycline). The tumor cells attached to the plate within 24-48 hours and dispersed into a monolayer over a period of 3 to 5 days. Once the cells became confluent, they were trypsinized using 0.25% Trypsin/EDTA and expanded onto 10cm plates. Upon completion of two passages, the cells were harvested and cryopreserved at −80° C. in freezing media (90% DMSO/10% FBS) for future use. Tumor cells that adapted to in vitro culture are referred to as BHc primary culture. The passage number of BHc primary culture is denoted with the letter “P” (e.g. BHc3_P4).

To test the conditional inducibility of oncogene expression in these cells in vitro, the cells were cultured in the presence or absence of inducer (doxycycline) and monitored for the modulation of oncogene expression as well as other characteristics such as cell morphology, proliferation rate.

To assess conditional tumorigenic status in vivo, the cells were cultured in the presence of doxycycline and injected subcutaneously into six immunocompromised mice (10⁶ cells per injection site, two sites per mouse). Doxycycline was administered to three of the six mice through food or water. The remaining three mice were maintained on food and water without doxycycline. The animals were monitored for tumor growth. Only the animals that received doxycycline developed tumors at the site of injection. After the tumors reached a volume of 500 mm³, the doxycycline was withdrawn from the food/water. This caused regression of the tumor growth, indicating that the cells of these tumors required the expression of the oncogene to remain tumorigenic. These conditionally tumorigenic BHc primary cultures are suitable for directed complementation, using a recombinant oncogene, as described in U.S. Patent Publication No. 2006/0228302.

BHc tumor archive: Although BHc primary cultures were successfully established, the in vitro culture condition might have selected for a biased population of cells that grow the best in non physiological conditions. In order to best preserve the properties of primary BHc tumors, these tumors were propagated in vivo. Briefly, primary BHc tumors were minced with scalpels and passed through 100 micron Cell Strainers (BD Biosciences) to remove debris. The cells were centrifuged at 1000 rpm for 5 minutes, washed twice with RPMI and counted using hemocytometer and resuspended in HBSS (Gibco Cat. No. 24020-117) and equal volume of Matrigel. 10⁴ to 10⁵ live cells (in 200 μl) were injected subcutaneously into ICR SCID mice and maintained on doxycycline food or water. Once the tumors reached 500-1000 mm³, they were collected and processed the same way as the primary BHc tumors. The resultant tumor cells could be passaged again in vivo or resuspended in freezing media (90% FBS [Gibco Cat. No. 10438-026]+10% DMSO), followed by serial temperature shift-down to liquid nitrogen storage for future use. Tumors expanded entirely in vivo without in vitro culture are referred to as archived BHc tumor. The passage number of archived BHc tumors is denoted with the letter “X” (e.g., BHc3_X2).

The conditional tumorigenecity of the BHc tumor archive was tested similar to the BHc primary culture. These archived BHc tumors were used for Directed Complementation in Example 4.

### Example 3

### Tight Regulation of CreERT² in BHc Primary Culture

CreERT2 was first transduced into HCT116 cells using a lentivirus. In these experiments, recombination between loxP sites, which resulted in excision of the recombinant gene of interest, was detected before (i.e., without) tamoxifen induction. Recombination in the absence of tamoxifen was attributed to excess CreER^{T2} production. To overcome the problem of CreER^{T2} overexpression in the BHc system, CreER^{T2} was inserted into the Rosa26 locus to achieve a predictable low level of expression. The regulation of Cre activity by tamoxifen was investigated in BHc primary culture quantitatively.

Retrovirus constructs: Retroviral vectors were used for transduction of both primary cultures and archived tumor cells prepared as described in Example 2 (above). The retrovirus backbone used in constructing all of the following retroviral vectors was pLHCX, which was obtained commercially (BD Biosciences Clontech, Palo Alto, Calif.).

pLGCD-1: The cDNA encoding hrGFP was PCR amplified from the Vitality phr-GFPII-1 vector (Strategene) and digested with EcoRI and BglII. pLHCX (Clontech) was digested with the same enzymes and the resulting 3.8 kb fragment was ligated with the hrGFP cDNA to produce pLGCX. The gateway destination cassette RfC.1 (Invitrogen) was inserted into pLGCX downstream of the CMV promoter to produce vector pLGCD-1. This is one of the backbone vectors used for Directed Complementation.

pLGCD-2: pLGCD-1 was modified as follows to make pLGCD-2, a backbone vector with the destination cassette flanked by loxP sites. The Destination Cassette in pLGCD-1 was amplified by PCR using primer YZ1010 5′-AGATCTAAGCTTATAACTTCGTATAGCATACATTATACGAAGTTATACAAGTTTGTA CAAAAAAG-3′ (SEQ ID NO: 2); and primer YZ1011 5′-CTCGAGATCGATAACTTCGTATAATGTATGCTATACGAAGTTATACCACTTTGTACA AGAAAG-3′ (SEQ ID NO: 3) to add loxP sites to both ends. The amplicon was digested with HindIII and ClaI, and ligated with the 6.2 kb fragment released from pLGCD-1 using the same enzymes, to produce pLGCD-2 (Clone YZ103.6). The PCR amplified region was sequenced to confirm that no undesirable mutations were introduced by PCR. The gene of interest in retroviral vectors based on pLGCD-2 can be excised upon activation of Cre.

pL-Her2^{YVMA}: The human Her2 cDNA was cloned into vector pENTR11 (Invitrogen, cat #11819-018) to generate vector pENTR11-Her2. A 12 nucleotide sequence, TACGTGATGGCA (SEQ ID NO: 4), which encodes peptide YVMA (SEQ ID NO: 5), was inserted into the Her2 kinase domain using the QuickChange II XL mutagenesis kit (Strategene) to produce vector pENTR11-Her2⁻^{^{YVMA}}. pLGCD-2 and pENTR11-Her2^{YVMA} were recombined through GATEWAY® LR reaction to produce vector pL-Her2^{YVMA}.

Pantropic retrovirus production: VSVG pseudotyped pantropic retrovirus was produced using the GP2-293 packaging cell line. GP2-293 cells were cultured in DMEM medium (containing 10% FBS, 50 U/ml penicillin, 50 μg/ml streptomycin) on 10 cm plates until 50-80% confluent. 6 μg of retroviral vector DNA was transfected into these cells using lipofectamine with plus reagent(Invitrogen). Medium from transfected plates were collected at 48 hours after infection and again 24 hours later. Virus particles in the medium were pelleted by ultra-centrifugation at 24,000 g for 2 hours. The pellets were resuspended in PBS at 4° C. overnight, then aliquoted and frozen at −80° C.

Infection: BHc tumor cells were cultured in DMEM medium (containing 10% FBS, 50 U/ml penicillin, 50 μg/ml streptomycin and 2 μg/ml doxycycline). At approximately 18-24 hours after plating, or when the plated cells were 70-80% confluent, the breast tumor cells were infected with 5 μl thawed pL-Her2^{YVMA} retroviral suspension in the presence of polybrene (8 μg/ml) overnight.

Tamoxifen induced Cre mediated recombination: The infected BHc primary cultures were expanded onto 6 cm plates. Once they became confluent again, each culture was split onto 2 6 cm plates. One of the two plates was treated with 1 uM 4-hydroxy-tamoxifen (4-OHT, Sigma) for 4 days. The other plate remained untreated. After the treatment, the cells were lysed and DNA was prepared using the Purigene DNA extraction Kit (Qiagen).

Cre mediated recombination was detected by quantitative PCR. Primers YZ1058 (5′-AATGGGCGTGGATAGCGGTTTG-3′) (SEQ ID NO: 6) and YZ1059 (5′-CCTACAGGTGGGGTCTTTCATTCC-3′) (SEQ ID NO: 7), which flank the loxP sites in pL-Her2^{YVMA}, can specifically detect the recombined product and will not amplify pL-Her2^{YVMA} in the unrecombined form. The primers YZ1060 (5′-CGGCCCCGTGATGAAGAAGA-3′) (SEQ ID NO: 8) and YZ1061 (5′-AGGCGGTGCTGGATGAAGTGGTA-3′) (SEQ ID NO: 9), which detect hrGFP, a region not affected by the recombination between the loxP sites, were used as loading control. Real time PCR was performed on ABI7900 and SYBR green was used to detect total DNA.

The quantitative PCR result demonstrated that Cre activity was tightly regulated in BHc tumor cells. No recombination was detected in cells that were not treated with 4-OHT. In all cultures that were treated with 4-OHT, recombination was clearly detected.

### Example 4

### Directed Complementation in archived BHc tumors

In vivo expansion of archived BHc tumor material: One vial of archived BHc tumor cells was thawed at 37° C. and 5 ml of warm DMEM medium was added. The live cells were counted by trypan blue exclusion. The cells were spun down and resuspended in HBSS to a concentration of 10⁶ cells/ml. Equal volume of matrigel (BD biosciences) was then mixed with cell suspension and 10⁵ cells (in 200 μl) were injected subcutaneously onto the flanks of ICR SCID mice. These mice were maintained on Doxycycline to support the growth of BHc tumors.

Infection: When BHc tumors reached 500 mm in size, the tumors were collected and processed as described in Example 2 for BHc tumor archive. After the number of live cells was counted, the cells were resuspended in RPMI medium (containing 10% FBS, 50 U/ml penicillin, 50 μg/ml streptomycin and 2 μg/ml doxycycline) to a concentration of 3×10⁵ cells/ml. 1 ml of tumors cells were infected with 30 μl of pantropic retrovirus in the presence of 8 μg/ml polybrene for 2 hours at 37° C. 1 ml of culture medium was added afterwards and the cells were further infected overnight.

Functional complementation: The next day, infected BHc tumor cells were trypsinized, rinsed and resuspended in HBSS Solution and equal volume of Matrigel. About 5×10⁴ infected tumor cells (in 200 μl) were injected into the flank of SCID mice maintained without doxycycline. The animals were observed for tumor development. Uninfected tumor cells were injected similarly as controls. Half of the control animals were maintained without doxycycline to monitor background tumor formation, the other half were maintained on doxycycline as quality control for the handling of tumor material.

Tumors complemented with pL-Her2^{YVMA} retrovirus developed after approximately 13 days in 6 out of 6 injection sites. The control mice on doxycycline developed tumors with the same latency and penetrance. No tumor was observed on mice injected with uninfected tumor cells that were maintained off doxycycline for more than 3 months. Tumors were harvested and tumor tissues were immediately snap-frozen in liquid nitrogen. RNA was isolated from tumor tissue and real-time PCR was performed to confirm the expression of target gene by using target gene specific primers. Human HER2 expression level in complemented tumors was about 10-15 fold higher than normal human reference, similar to doxycycline-induced tumors. The expression of retroviral constructs in tumor cells was also confirmed by GFP protein immunohistochemistry on formalin fixed tumor samples.

### Example 5

### Dependence of DC tumors on Virally Expressed Complementing Oncogene

DC tumor collection and propagation: Directed Complementation (DC) tumors were propagated in vivo. About 0.2 g of surgically resected direct complemented tumor was minced and resuspended in freezing media (90% FBS [Gibco Cat. No. 10438-026]+10% DMSO), followed by serial temperature shift-down to liquid nitrogen storage for future use.

Minced DC tumor was thawed at 37° C. and cells were dissociated by passing through cell strainers (100 μm filters, BD Falcon Cat. No. 352360). The cells were collected and centrifuged at 1000 rpm for 5 minutes, washed twice with PBS and counted using hemocytometer and resuspended in one part PBS (Gibco Cat. No. 24020-117) to one part Matrigel (BD Cat. No. 3542334) for injections. When 0.1 million cells were injected subcutaneously into immunocompromised mice, tumors were observed in about 7-10 days.

Tamoxifen treatment: To activate CreER^{T2} and induce Cre mediated recombination (i.e., excision) between loxP sites, animals were treated with 0.1 ml tamoxifen (10 mg/ml in corn oil) through intraperitoneal (IP) injection for five consecutive days.

A four-pronged xenograft experiment was carried out to demonstrate conclusively that continuous expression of encoded by the retroviral construct is required for tumor maintenance of the DC tumors generated in Example 4, and that Her2^{YVMA} indeed can functionally complement the switched-off Her2^{neu} gene in BHc tumors and restore tumorigenicity.

About 0.1 million DC tumor cells were injected subcutaneously into 10 NCR nude mice on the right flank. These mice were maintained off doxycycline. Similarly 10 NCR nude mice were inoculated with archived BHc tumor and maintained on doxycycline. These mice were divided into 4 groups.

The first group contained of 5 mice bearing BHc tumors. They were not treated with Tamoxifen. Tumors were observed on these mice within 10 days.

The second group contained of 5 mice bearing BHc tumors. They were treated with Tamoxifen for 8 consecutive days. Although tumors on these mice grew slower than group 1, nonetheless, tumors were observed on all 5 mice within 12 days and they continued to grow.

The third group contained of 5 mice bearing DC tumors. They were not treated with Tamoxifen. Tumors were observed on these mice within 10 days.

The fourth group contained of 5 mice bearing DC tumors. They were treated with Tamoxifen for 8 consecutive days. Tumors were never observed on these mice (up to a month until the experiment was terminated).

These results show that the Cre-ER system functions as designed to assist in the identification of tumor maintenance genes.

We claim:

1. A method of producing a tumorigenic mouse cell, the tumorigenicity of which depends on expression of a recombinant gene of interest, comprising the steps of:
(a) providing a conditionally tumorigenic mouse cell comprising
(i) one or more mutations such that both alleles of an endogenous tumor suppressor gene are absent or nonfunctional,
(ii) a gene construct encoding a Cre-ER fusion protein, wherein the gene construct is operatively linked to an endogenous Rosa26 promoter, and
(iii) a recombinant oncogene operably linked to a tetracycline inducible promoter, wherein
(1) expression of the recombinant oncogene results in tumorigenicity of the conditionally tumorigenic mouse cell, and
(2) the tetracycline inducible promoter is in the uninduced state; and
(b) introducing into the cell a recombinant gene of interest flanked by loxP sites that functionally complements the recombinant oncogene thereby restoring tumorigenicity of the cell without expression of the recombinant oncogene.

2. The method of claim 1, wherein the tumor suppressor gene is selected from the group consisting of Rb, P53, INK4a , PTEN, LATS, Apaf1, Caspase 8, APC, DPC4, KLF6, GSTP1, ELAC2/HPC2, NKX3.1, ATM, CHK2, ATR, BRCA1, BRCA2, MSH2, MSH6, PMS2, Ku70, Ku80, DNA/PK, XRCC4, Neurofibromatosis Type 1, Neurofibromatosis Type 2, Adenomatous Polyposis Coli, the Wilms tumor-suppressor protein, Patched and FHIT.

3. The method of claim 2, wherein the tumor suppressor gene is selected from the group consisting of INK4a , P53, PTEN, and Rb.

4. The method of claim 3, wherein the tumor suppressor gene is INK4a .

5. The method of claim 1, wherein the recombinant oncogene is selected from the group consisting of Her2, KRAS, HRAS, NRAS, EGFR, FGFR1, FGFR2, FGFR3, FGFR4, MDM2, TGF-β, RhoC, AKT, c-myc, β-catenin, PDGF, C-MET, PI3K-CA, CDK4, cyclin B1, cyclin D1, estrogen receptor alpha gene, progesterone receptor gene, ErbB1, ErbB3, PLK3, KIRREL, ErbB4, TGFα, ras-GAP, Shc, Nck, Src, Yes, Fyn, Wnt, Bcl2, PyV MT antigen, and SV40 T antigen.

6. The method of claim 5, wherein the recombinant oncogene is selected from the group consisting of Her2, KRAS, C-MET, PI3K-CA and AKT.

7. The method of claim 6, wherein the recombinant oncogene is Her2 or KRAS.

8. The method of claim 1, wherein the gene of interest is selected from the group consisting human AKT1, human EGFR*, and human mTOR.

9. A tumorigenic mouse cell produced by the method of claim 1.

10. A method of identifying a tumor maintenance gene, comprising the steps of:
a) producing, according to the method of claim 1, a multiplicity of tumorigenic mouse cells, the tumorigenicity of which depends on the expression of a recombinant gene of interest;
b) implanting at least one tumorigenic mouse cell into a host mouse;
c) obtaining in the host mouse a tumor derived from the implanted cells;
d) administering an exogenous antiestrogen to the mouse; and
e) determining the effect, if any, of the loss of expression of the recombinant gene of interest on the tumor, wherein a decrease in the size of the tumor is indicative that the gene of interest is a tumor maintenance gene.

11. The method of claim 10, wherein the antiestrogen is selected from the group consisting of tamoxifen, 4-hydroxytamoxifen, and RU486.

12. The method of claim 10, wherein the antiestrogen is tamoxifen.

13. The method of claim 10, wherein the antiestrogen is administered following tumor formation.

14. The method of claim 10, wherein the antiestrogen is administered at the time of cell implantation.

15. The method of claim 10, wherein the loss of expression of the recombinant gene of interest results in a loss of tumorigenicity of the conditionally tumorigenic mouse cells.
